(19) United States
(12) Patent Application Publication
(10) Pub. No.: US 2013/0297031 A1
(43) Pub. Date: Nov. 7, 2013

(54) PATIENT SPECIFIC INSTRUMENTS AND RELATED METHODS FOR JOINT REPLACEMENT

(71) Applicant: CONFORMIS, INC., Bedford, MA (US)

(72) Inventor: Mahmoud A. Hafez, Cairo (EG)

(73) Assignee: CONFORMIS, INC., Bedford, MA (US)

(21) Appl. No.: 13/886,040

(22) Filed: May 2, 2013

Related U.S. Application Data
(60) Provisional application No. 61/641,851, filed on May 2, 2012.

(51) Int. Cl. A61F 2/30

(52) U.S. Cl. CPC A61F 2/30942
USPC 623/20.14

(57) ABSTRACT

Devices, tools and techniques for the design, selection and/or modification of patient-specific implants, instruments and related methods are disclosed. Various embodiments include the use of double joint lines, particularly for treating severe deformities during joint repair and/or replacement surgeries.

### RELATED APPLICATIONS

This application claims the benefit of U.S. Provisional Application Ser. No. 61/641,851, entitled “Patient Specific Instruments and Related Methods for Joint Replacement” and filed May 2, 2012, the disclosure of which is incorporated herein by reference in its entirety.

### TECHNICAL FIELD

This disclosure relates to devices, tools and techniques for the design, selection and/or modification of patient-specific implants, instruments and related methods, particularly for treating severe deformities during joint repair and/or replacement surgeries.

### BACKGROUND

In traditional joint replacement surgeries, especially those involving the repair and/or replacement of a significant portion of a diseased or damaged joint structure (e.g., a “total joint” surgery), a surgeon typically has a limited number of implant sizes and/or shapes from which to select an appropriate joint replacement implant. In general, the available implant components will be designed and intended to accommodate a relatively large subgroup of a given patient population, and it is the surgeon's challenge to alter the patient's natural anatomy to a sufficient degree to accommodate one or more of the available implants. Typically, such implant components are designed to replicate and/or accommodate an average or more “normalized” patient anatomy.

Moreover, in typical surgical practice, a surgeon's surgical objective will often be to create a more “normal” anatomy for the repaired joint structure, which may be due to one or more factors, including because (1) the available implant components require a normalized anatomical support structure, (2) the available repair components are designed and/or tested to only recreate and/or replicate more normalized anatomical structures and/or joint motion, and/or (3) the surgeon is familiar with and comfortable with more normalized joint motion, and thus he or she attempts to create such “normal” motion within the repaired anatomical structures.

### SUMMARY

According to certain embodiments, a method of making an implant component for a knee joint of a patient is disclosed that includes deriving information regarding a first joint line of the joint based, at least in part, on patient-specific information. The method also includes determining a planned level of resection for a first portion of a bone of the joint based, at least in part, on the patient-specific information. Further, a dimension of the implant component is determined based, at least in part, on the derived information regarding the first joint line and the planned level of resection for the first portion of the bone.

According to certain embodiments, an implant component for treating a patient's joint is disclosed that includes a medial bone-facing surface. The medial bone-facing surface is positioned to engage a cut bone surface of a medial portion of a proximal tibia at a first level. The implant component also includes a lateral bone-facing surface. The lateral bone-facing surface is positioned to engage a cut bone surface of a lateral portion of the proximal tibia at a second level. The first level is offset from the second level. The implant component additionally includes one or more joint-facing surfaces having a curvature based, at least in part, on patient-specific information.

According to certain embodiments, a system for treating a joint of a patient is disclosed that includes one or more patient-specific instruments. The system further includes a medial tibial implant component. The medial tibial implant component has a bone-facing surface and a joint-facing surface. The joint-facing surface has a curvature based, at least in part, on patient-specific information. The system also includes a lateral tibial implant component, which has a bone-facing surface and a joint-facing surface. The joint-facing surface of the lateral tibial implant has a curvature based, at least in part, on patient-specific information. The bone-facing surface of the medial tibial implant component is configured to engage a cut bone surface that is at a level offset from the level of a cut bone surface to which the bone-facing surface of the lateral tibial implant component is configured to engage. The system further includes a femoral implant component, which has a joint-facing surface with a curvature based, at least in part, on patient-specific information.

According to certain embodiments, patient-specific instruments that can be used for double joint line knee joint replacement surgical procedures are disclosed.

### BRIEF DESCRIPTION OF THE DRAWINGS

Objects, aspects, features, and advantages of various embodiments will become more apparent and may be better understood by referring to the following description, taken in conjunction with the accompanying drawings, in which:

FIG. 1 depicts a coronal view of three knee joints relative to a native joint line;

FIG. 2 depicts a lateral view of three knee relative to a native joint line;

FIG. 3 provides two images on the left showing severe varus deformity and an image on the right showing two knee joints in which varus deformity has been corrected through bilateral joint replacement;

FIG. 4 depicts a coronal view of exemplary embodiments of joint lines for a knee joint with femoral lateral bone loss in extension and a knee joint with femoral medial bone loss in extension;

FIG. 5 depicts a coronal view of exemplary embodiments of joint lines for the knee joints of FIG. 4 in flexion;

FIG. 6 depicts a coronal view of exemplary embodiments of joint lines for a knee joint with significant tibial lateral bone loss in extension and a knee joint with significant tibial medial bone loss in extension; and

FIG. 7 depicts a coronal view of exemplary embodiments of joint lines for the knee joints from FIG. 6 in flexion.

Additional figure descriptions are included in the text below. Unless otherwise denoted in the description for each figure, “M” and “L” in certain figures indicate medial and lateral sides of the view, respectively; “A” and “P” in certain figures indicate anterior and posterior sides of the view, respectively; and “S” and “I” in certain figures indicate superior and inferior sides of the view, respectively.

### DETAILED DESCRIPTION

In this application, the use of the term “including,” as well as other forms, such as “includes” and “included,” is not limiting. Also, terms such as “element” or “component” encompass both elements and components comprising one unit and elements and components that comprise more than one subunit, unless specifically stated otherwise. In addition, the use of the term “portion” may include part of a moiety or the entire moiety.

Additionally, in this application, use of the terms “implant” and “implant component” encompass both an implant and/or component that is one of multiple implants or components making up a single implanted structure and an implant or component that constitutes the entire implanted structure. Further, an “implant system” can include one or more implant components and, optionally, one or more related surgical tools.

Often, surgeons encountering unusual and/or severe joint deformity in a patient attempt to surgically “normalize” the joint anatomy in various ways, including the use of surgical resection strategies and/or implant components to recreate a more “normalized” joint structure and/or function. However, for patient's having unusual and/or severe joint deformity, it may be counterproductive to “normalize” and/or otherwise significantly alter the patient's pre-existing joint alignment, motion, spacing, orientation and/or kinematics. Rather, it may be desirous for a surgeon to employ a surgical strategy that is to some degree particularized to the patient (either the individual patient and/or a group of patients having similar levels of a similar deformity), and which may include resection strategies, surgical tools and/or surgical implant components that are designed using, at least in part, patient-specific anatomical data, and which seek to replicate and/or accommodate the patient's particular deformities and/or pre-existing joint alignment, motion, spacing, orientation and/or kinematics.

In various embodiments, patient-specific implants, tools, and/or related methods or systems can be developed by methods that can include obtaining and analyzing imaging data of the patient's joint(s) and pre-operatively developing a surgical plan (including, e.g., selecting and/or designing implant components and tools, alignment, positioning). The imaging test data can include, for example, data generated from CT scans. Further, in some embodiments, rapid prototyping can be used to manufacture at least some of the tools based on the patient-specific information.

In at least one preferred embodiment, a surgical plan for addressing a joint having an unusual and/or severe joint deformity can include implants, tools and surgical procedures that desirably assess the medial and lateral compartments (or other individual features) of a joint on an individual basis, which can result in surgical implant components and tools/procedures particularized to an individual patient and/or group of patients having similar deformities. For example, in the case of a severely deformed knee joint, a surgical plan and associated tools/implants may be designed, selected and/or created employing dual or “double joint lines,” with each “joint line” (and/or joint plane) being assessed between the femoral and tibial articulating surfaces of an individual medial or lateral compartment of the knee. In this manner, a surgical plan can be created that minimizes unnecessary resection of relevant anatomical support structures, while maintaining and/or adapting the “deformed” anatomy and/or kinematics, in an appropriate manner, to the intended joint replacement.

There are 3 joint lines in the native knee: proximal tibial, distal femoral and posterior femoral. The joint line of the tibia inclines about 3° to the tibial shaft as from lateral to medial and has a posterior sloping of about 5° as moving from the front to the back of the knee. The tibial joint line lies above fixed bony landmarks such as tibial tuberosity or the fibular head and its level can be measured in mm distance from these landmarks. The inclination of the distal femoral joint line to the femoral shaft is about and varies from 7° to 11° . The level of femoral joint line can also be measured as the distance to certain anatomical landmarks such as epicondylar eminences. This level should be considered in extension and also in flexion (posterior femoral line), it has been reported that the absolute distance from bony landmarks to the femoral joint line is unreliable as there are vast differences between individuals in these landmarks. A linear correlation between the width of the trans-epicondylar axis and the perpendicular distance from the epicondyles to the joint-line tangent may be determined. This ratio is useful to calculate the true joint-line position in revision total knee arthroplasty (TKA). So, the epicondylar ratio was made (distance from lateral epicondyle to the joint line divided by femoral width) averaged 28% (Servien E, Viskontas D, Giuffre B M, Coolican M R, Parker D A. Reliability of bony landmarks for restoration of the joint line in revision knee arthroplasty, Knee Surg Sports Traumatol Arthrosc. 2008 March; 16(3):263-9; Romero J, Seifert B, Reinhardt O, Ziegler O; Kessler O. A useful radiologic method for preoperative joint-line determination in revision total knee arthroplasty. Clin Orthop Relat Res. 2010 May; 468(5):1279-83).

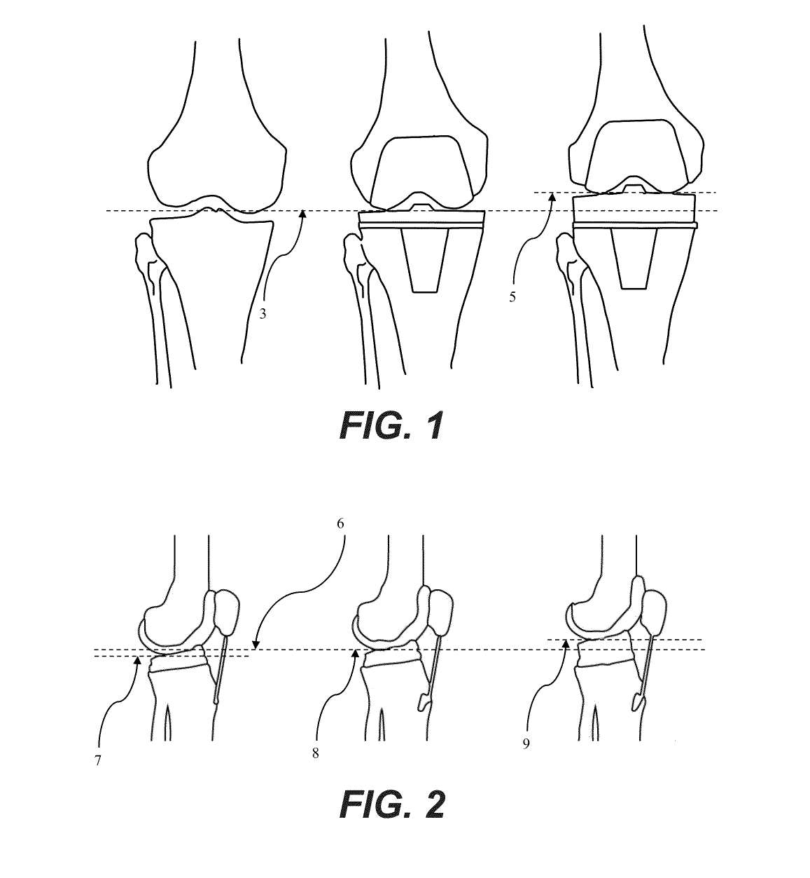

The level of joint line will desirably be maintained or be kept as dose to normal while performing total knee arthroplasty to allow normal kinematics, soft tissue balance and stability. Conventional techniques of TKA do not follow the normal anatomy of the tibial or femoral joint lines. The classic method described by Freeman and Insall produce a 3° varus malalignment while making the distal femoral cut, which is compensated by another 3° of valgus malalignment for the tibial cut. Although this method maintains the relationship between tibial and femoral joint line in extension, it requires another compensatory malalignment cut (3° external rotation) of the posterior femur. These malalignment and compensatory cuts can lead to changes in the morphology; and possibly the function of the knee joint by removing unequal amount of bone from the medial and lateral aspects of the tibia. The same can happen for the femur, but at 2 reference cuts, namely the distal and antero-posterior. FIG. 1 shows the joint lines in coronal view in the native knee, in TKA when it is maintained 3, and when it is elevated 5. FIG. 2 shows the joint lines in lateral views in TKA when it is lowered 7, maintained 8, and elevated 9, relative to the native joint line.

In a posterior stabilized TKA (PCL sacrificing), there can be a high risk of elevating the joint line. Elevation greater than 8 mm can significantly affect the knee kinematics and may result in PF problems and the need for revision. Retention of the PCL also requires strict maintenance of the joint line (Aaron G Rosenberg, Donald M Knapke. Posterior cruciate retaining total knee arthroplasty. In Surgery of the Knee (4th Ed.). Install J N, Scott N (Eds). Philadelphia, Churchill Livingston. 2006, 1522-1530). More difficulties in maintaining the normal joint lines are observed in revision TKA, when the anatomical landmarks that guide the surgeon to the normal level of joint lines are usually lost.

Another problem with joint line and the excessive amount of bone resection can particularly occur during TKA for severe articular deformities (valgus or varus). Using conventional techniques to maintain joint lines, surgical resection strategies usually lead to excessive bone resection in one side of the tibia or femur. For example, in severe varus malalignments, excessive tibial cuts can occur in the lateral side, significantly and undesirably compromising the bone stock. FIG. 3 shows one example of a severe varus deformity, and the amount of bone that may be removed from a lateral side of the joint to restore the joint line and make it leveled at the medial and lateral sides.

Accordingly, the level and the inclination of the normal joint lines of the knee may be difficult to maintain during primary and revision TKA. Attempts to maintain a normalized joint line may come at the expense of the bone stock when more cuts are done on one side of the tibia (either medial and/or lateral) and similar in both distal and antero-posterior femoral cuts.

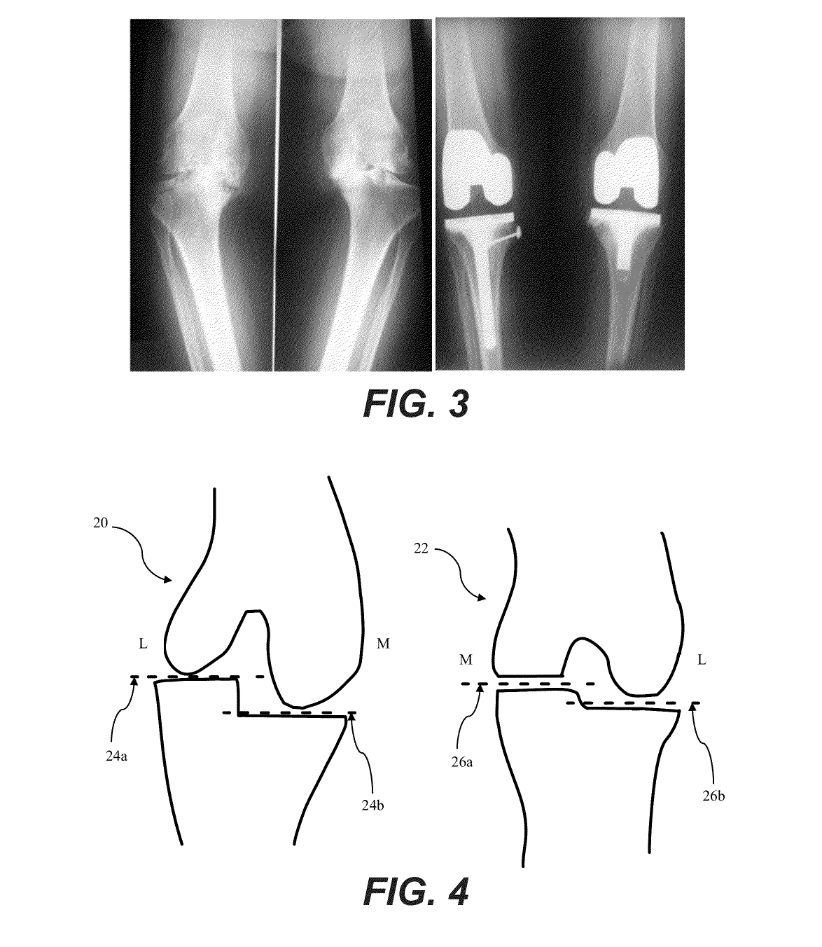

In some embodiments, the use of double joint lines can be used in the selection and/or design of various aspects of a surgical plan to treat a deformed joint. FIG. 4 illustrates a knee joint 20 with femoral lateral bone loss in extension, as compared to a knee joint 22 with femoral medial bone loss in extension, and embodiments of double joint lines for use in treating the joints. For example, joint lines 24a and 24b can be used on the lateral and medial compartments, respectively, in treatment of the knee joint 20. Similarly, joint lines 26a and 26b can be used on the medial and lateral compartments, respectively, in treatment of the knee joint 22. The use of various combinations of double joint lines (e.g., 24a and 24b, 26a and 26b), including as described above, can be created or maintained and utilized in conjunction with the design and/or selection of tibial implants having differing thicknesses on the lateral side vs. medial side.

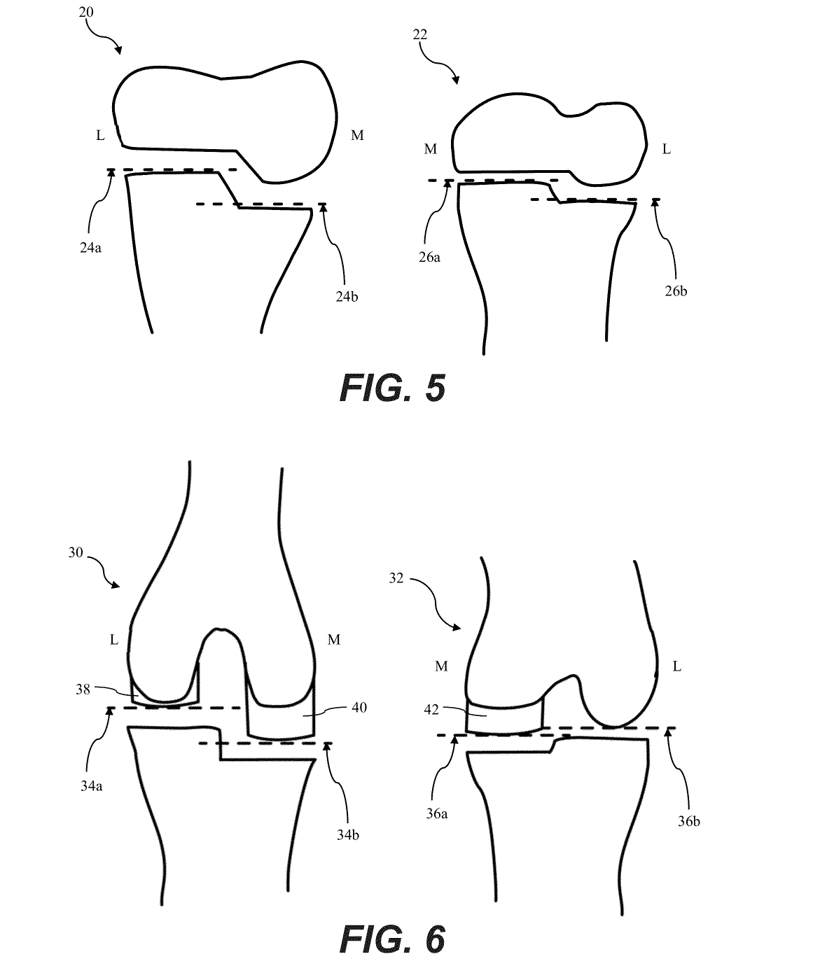

FIG. 5 illustrates the exemplary embodiments of joint lines for the knee joints 20 and 22 of FIG. 4, in flexion. As shown, for knee joint 20, with femoral lateral bone loss, a thicker lateral tibial implant can be used on the knee joint to accommodate the femoral lateral bone loss. As similarly shown, for knee joint 22, with femoral medial bone loss, a thicker medial tibial implant can be used on the knee joint to accommodate the femoral medial bone loss.

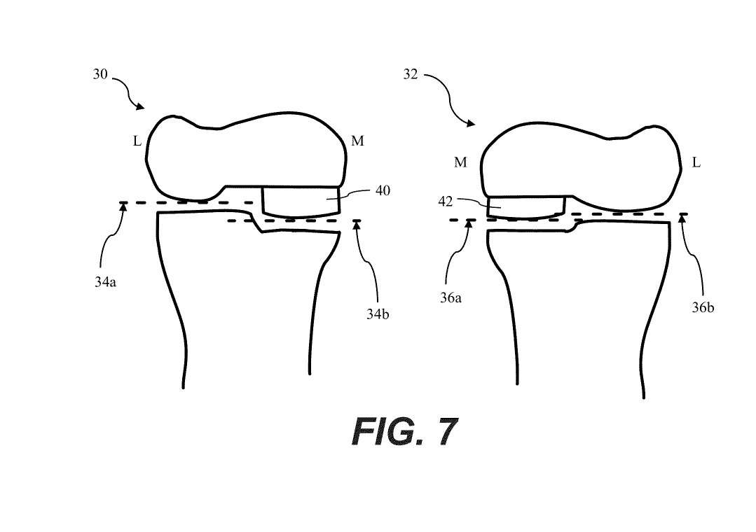

FIG. 6 illustrates a knee joint 30 with significant tibial lateral bone loss in extension, as compared to a knee joint 32 with significant tibial medial bone loss in extension. In some embodiments, joint lines 34a and 34b can be used on the lateral and medial compartments, respectively, in conjunction with femoral implants 38 and 40 for treatment of a knee joint with tibial lateral bone loss in extension. While for treatment of knee joints with significant tibial medial bone loss in extension, joint lines 36a and 36b can be used on the medial and lateral compartments, respectively, in conjunction with femoral implants 42 for treatment. FIG. 7 illustrates the embodiments for treatment of the knee joints of FIG. 6, in flexion.

It is to be understood that the features of the various embodiments described herein are not mutually exclusive and may exist in various combinations and permutations.

What is claimed is:

1. A method of making an implant component for a knee joint of a patient, the method comprising:
providing patient-specific information associated with the joint;
deriving information regarding a first joint line of the joint based, at least in part, on the patient-specific information;
determining a planned level of resection for a first portion of a bone of the joint based, at least in part, on the patient-specific information; and
deriving a dimension of the implant component based, at least in part, on the derived information regarding the first joint line and the planned level of resection for the first portion of the bone.

2. The method of claim 1, further comprising deriving a joint-facing surface of the implant component having a shape, which includes a patient-specific curvature, based on the patient-specific information.

3. The method of claim 1, wherein the patient-specific information comprises information derived from a CT scan of the joint.

4. The method of claim 1, wherein the patient-specific information comprises anatomical data.

5. The method of claim 1, wherein the joint comprises a deformed knee joint.

6. The method of claim 1, wherein the first joint line comprises a joint line between articulating surfaces of a medial compartment or of a lateral compartment of the joint.

7. The method of claim 1, wherein the first joint line comprises a joint line between articulating surfaces of a medial compartment of the joint, and wherein a level of the first joint line differs from a level of a joint line between articulating surfaces of a lateral compartment of the joint.

8. The method of claim 1, wherein the first joint line comprises a joint line between articulating surfaces of a lateral compartment of the joint, and wherein a level of the first joint line differs from a level of a joint line between articulating surfaces of a medial compartment of the joint.

9. The method of claim 1, wherein the information regarding the first joint line comprises a level of the first joint line.

10. The method of claim 1, wherein the deriving information regarding the first joint line comprises determining a level of the first joint line based, at least in part, on a width of a trans-epicondylar axis of the joint.

11. The method of claim 1, further comprising:
deriving information regarding a second joint line of the joint based, at least in part, on the patient-specific information;
determining a planned level of resection for a second portion of the bone of the joint based, at least in part, on the patient-specific information; and
deriving a dimension of the implant component based, at least in part, on the derived information regarding the second joint line and the planned level of resection for the second portion of the bone.

12. The method of claim 11, wherein the first joint line comprises a joint line between articulating surfaces of a medial compartment of the joint, and wherein the second joint line comprises a joint line between articulating surfaces of a lateral compartment of the joint.

13. The method of claim 11, wherein the first potion of a bone of the joint comprises a medial portion of a proximal tibia of the joint.

14. The method of claim 11, wherein the second portion of a bone of the joint comprises a lateral portion of a proximal tibia of the joint.

15. The method of claim 11, wherein the planned level of resection for the first portion of the bone of the joint differs from the planned level of resection for the second portion of the bone of the joint.

16. The method of claim 11, further comprising designing a first bone-facing surface of the implant component such that the first bone-facing surface is configured to engage the bone of the joint at the planned level of resection for the first portion of the bone and designing a second bone-facing surface of the implant component such that the second bone-facing surface is configured to engage the bone of the joint at the planned level of resection for the second portion of the bone.

17. An implant component for treating a patient's joint, comprising:
a medial bone-facing surface, the medial bone-facing surface configured to engage a cut bone surface of a medial portion of a proximal tibia at a first level;
lateral bone-facing surface, the lateral bone-facing surface configured to engage a cut bone surface of a lateral portion of the proximal tibia at a second level; and
one or more joint-facing surfaces having a curvature based, at least in part, on patient-specific information,
wherein the first level is offset from the second level.

18. The implant component of claim 17, wherein the implant component comprises a tibial tray, and wherein the tibial tray includes the medial and lateral bone-facing surfaces.

19. A system for treating a joint of a patient, the system comprising:
one or more patient-specific instruments;
a medial tibial implant component, the medial tibial implant component including a bone-facing surface and a joint-facing surface, the joint-facing surface having a curvature based, at least in part, on patient-specific information;
a lateral tibial implant component, the lateral tibial implant component including a bone-facing surface and a joint-facing surface, the joint-facing surface having a curvature based, at least in part, on patient-specific information; and
a femoral implant component, the femoral implant component including a joint-facing surface having a curvature based, at least in part, on patient-specific information,
wherein the bone-facing surface of the medial tibial implant component is configured to engage a cut bone surface that is at a level offset from the level of a cut bone surface to which the bone-facing surface of the lateral tibial implant component is configured to engage.

20. The system of claim 19, wherein a level of the joint-facing surface of the medial tibial implant component is configured to be offset from the level of the joint-facing surface of the lateral tibial implant component when implanted.
